(19) United States
(12) Patent Application Publication
(10) Pub. No.: US 2015/0032382 A1
(43) Pub. Date: Jan. 29, 2015

(54) APPARATUS AND METHOD FOR DETERMINING DROWSY STATE

(71) Applicant: HYUNDAI MOTOR COMPANY, Seoul (KR)

(72) Inventors: Hwal Ly LEE, Seoul (KR); Jin Kwon KIM, Gwangmyeong-si (KR); Min Cheol WHANG, Goyang-si (KR)

(73) Assignee: HYUNDAI MOTOR COMPANY, Seoul (KR)

(21) Appl. No.: 14/060,254

(22) Filed: Oct. 22, 2013

(30) Foreign Application Priority Data
Jul. 24, 2013 (KR) 10-2013-0087518

(51) Int. Cl. A61B 5/18

(52) U.S. Cl. CPC A61B 5/18
USPC 702/19; 600/301

(57) ABSTRACT

An apparatus and a method for determining a drowsy state include an information acquirer acquiring physiological information of a user, a mode determiner setting an operation mode among modes categorized according to variable applied to determine a drowsy state of the user. A threshold determiner sets a threshold for an application variable of the set mode based on data in a reference table stored in database (DB). A physiological index calculator calculates a measurement value for each of the application variables based on state information of the user acquired in real time from the information acquirer. A state determiner determines the drowsy state of the user by comparing a user table generated from the measurement value calculated for each of the application variables with the reference table.

### CROSS-REFERENCE TO RELATED APPLICATION

This application claims the benefit of priority to Korean Patent Application No. 10-2013-0087518, filed on Jul. 24, 2013 in the Korean Intellectual Property Office, the disclosure of which is incorporated herein in its entirety by reference.

### TECHNICAL FIELD

The present disclosure relates to an apparatus and a method for determining a drowsy state, and more particularly, to a technology for determining a drowsy state based on a change in a physiological state of a driver.

### BACKGROUND

Generally, there is a method using a physiological signal to determine a drowsy state of a driver.

The method for determining drowsy driving by using the physiological signal is to determine the drowsy state of the driver by analyzing information for a specific variable that may be obtained from the physiological signal. However, since the variable applied to determine the drowsy state is limited to some of the variables, there is a limitation in considering the overall situations of the driver.

In addition, since the determination of the drowsy state is independently determined and not comprehensively considered with respect to each of the specific variables, the drowsy state of the driver cannot be analyzed in detail.

### SUMMARY

The present disclosure has been made in view of the above problems and provides an apparatus and a method for determining a drowsy state capable of determining the drowsy state of a user in real time by comparing a reference table prepared based on a plurality of users with a user table prepared according to a change in a physiological state of a user.

The present disclosure provides an apparatus and a method for determining a drowsy state capable of simply and rapidly determining the drowsy state of a user by comparing with the reference table based on physiological information, action information and the like acquired in real time from a user during driving of a vehicle.

The present disclosure further provides an apparatus and a method for determining a drowsy state capable of precisely determining the drowsy state of a user by comprehensively considering a state change for whole variable or valid variables that may be used for determining the drowsy state of the user.

In accordance with an exemplary embodiment of the present disclosure, an apparatus for determining a drowsy state includes an information acquirer acquiring physiological information of a user. A mode determiner sets an operation mode among modes categorized according to a variable applied to determine the drowsy state of the user. A threshold determiner sets a threshold for an application variable of the set mode based on data in a reference table stored in database (DB). A physiological index calculator calculates a measurement value for each of the application variables based on state information of the user acquired in real time from the information acquirer. A state determiner determines the drowsy state of the user by comparing a user table generated from the measurement value calculated for each of the application variables with the reference table.

The application variable includes at least one among electroencephalogram (EEG), photoplethysmograph (PPG), photoplethysmograph rate variability (PRV), galvanic skin response (GSR), skin temperature (SKT), electrooculography (EOG), and respiration (RSP).

The reference table is generated according to a change in a physiological signal value measured in the drowsy state based on a physiological signal value measured in a state of awake from a plurality of users with respect to variables applied to determine the drowsy state of the user.

The user table is generated from results obtained by comparing the measurement value calculated for each of the application variables with a reference value for each of the application variables.

The reference value is a mean value of the value measured for a given time from a time point of starting to measure a physiological signal of the user.

The reference value is set based on data measured in an awake state among the data in the reference table.

The mode determiner sets any one of a first mode operating only by presetting some valid variables among the application variables and a second mode operating for the whole application variables as the operation mode.

The valid variable is a variable that has a change of a physiological signal value exceeding a standard value when the physiological signal value is measured in the drowsy state based on the physiological signal value measured in an awake state among the application variables.

The information acquirer acquires together with action information of the user.

In accordance with another exemplary embodiment of the present disclosure, a method for determining a drowsy state includes acquiring physiological information of a user. An operation mode is set among modes categorized according to a variable applied to determine the drowsy state of the user. A threshold for an application variable of the set mode is set based on data in a reference table stored in database (DB). A measurement value for each of the application variables is calculated based on physiological information acquired in real time from the user. A user table is generated by comparing the measurement value calculated for each of the application variables with a reference value for each of the application variables. The reference table called from the DB is compared with the user table and the drowsy state of the user is determined according to the comparison result.

Before acquiring the physiological information of the user, the reference table is generated according to a change of a physiological signal value measured in the drowsy state based on a physiological signal value measured in an awake state from a plurality of users with respect to variables applied to determine the drowsy state of the user.

### BRIEF DESCRIPTION OF THE DRAWINGS

The objects, features, and advantages of the present disclosure will be more apparent from the following detailed description in conjunction with the accompanying drawings.

FIG. 1 is a block diagram illustrating a configuration of an apparatus for determining a drowsy state according to an exemplary embodiment of the present disclosure.

FIG. 2 is a diagram illustrating a reference table according to an exemplary embodiment of the present disclosure.

FIG. 3 is a diagram for describing an operation of an apparatus for determining a drowsy state according to an exemplary embodiment of the present disclosure.

FIGS. 4 and 5 are flow charts illustrating a flow of operation for a method for determining a drowsy state according to an exemplary embodiment of the present disclosure.

### DETAILED DESCRIPTION

Exemplary embodiments of the present disclosure are described with reference to the accompanying drawings in detail. The same reference numbers are used throughout the drawings to refer to the same or like parts. Detailed descriptions of well-known functions and structures incorporated herein may be omitted to avoid obscuring the subject matter of the present disclosure.

An apparatus for determining a drowsy state according to an exemplary embodiment of the present disclosure is provided in a vehicle and determines the drowsy state in real time according to a change in physiological state of a driver driving the vehicle to prevent a dangerous situation due to drowsy driving in advance.

Hereinafter, exemplary embodiments of the present disclosure will be described in detail with reference to the accompanying drawings.

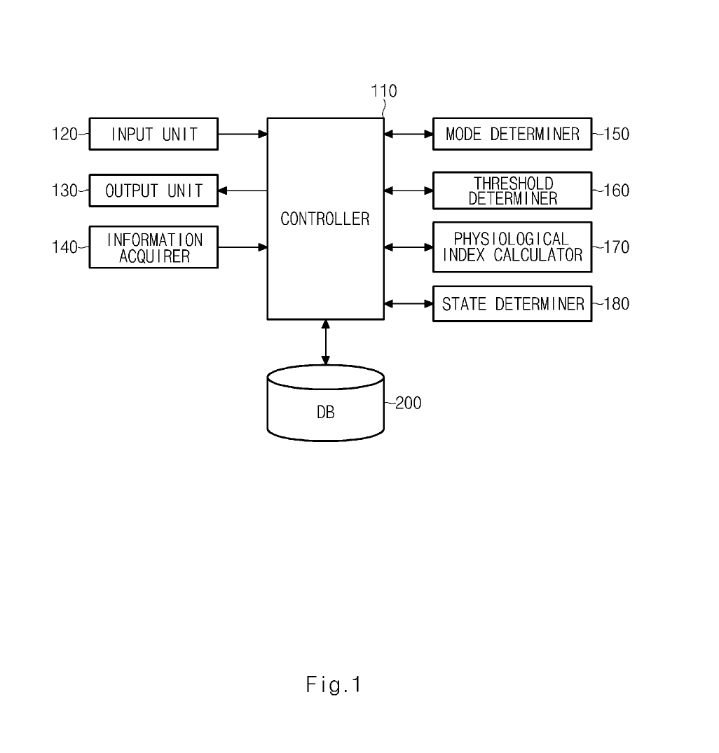

FIG. 1 is a block diagram illustrating a configuration of an apparatus for determining a drowsy state according to an exemplary embodiment of the present disclosure. Referring to FIG. 1, the apparatus for determining a drowsy state according to an exemplary embodiment of the present disclosure includes a controller 110, an input unit 120, an output unit 130, an information acquirer 140, a mode determiner 150, a threshold determiner 160, a physiological index calculator 170, a state determiner 180, and database (DB) 200. Here, the controller 110 controls the operation of each unit of the apparatus for determining the drowsy state.

The input unit 120, which is a means to input information and control commands to operate the apparatus for determining the drowsy state, may be provided in an instrument panel of a vehicle.

The output unit 130 may output an operating state, processing results, and the like of the apparatus for determining the drowsy state and may output alarm information according to a drowsy state determination result. In this case, the output unit 130 may be a displaying means such as a monitor provided in the instrument panel of the vehicle or navigation screen and may also be a voice outputting means such as a speaker or a buzzer.

The information acquirer 140 may acquire physiological information, action information, and the like of the user. As an example, the information acquirer 140 may acquire a signal from brain wave, pulse wave, heart, skin, and the like and may acquire the action information such as eyeball movement, yawning, and the like of the driver.

In the DB 200, an algorithm for determining the drowsy state may be stored. In addition, in the DB 200, a reference table applied to the algorithm for determining the drowsy state may be stored. In this case, the DB 200 may be provided in the apparatus for determining the drowsy state and may be a storing device provided at the outside thereof.

The reference table is generated according to a change in a physiological signal value measured in the drowsy state based on a physiological signal value measured in an awake state by using the physiological signal acquired in real time from a plurality of users. In this case, the reference table records the change of the physiological signal value for each of variables applied to determine the drowsy state of the plurality of users.

The variable applied to determine the drowsy state corresponds to at least one among electroencephalogram (EEG), photoplethysmograph (PPG), photoplethysmograph rate variability (PRV), galvanic skin response (GSR), skin temperature (SKT), electrooculography (EOG), and respiration (RSP).

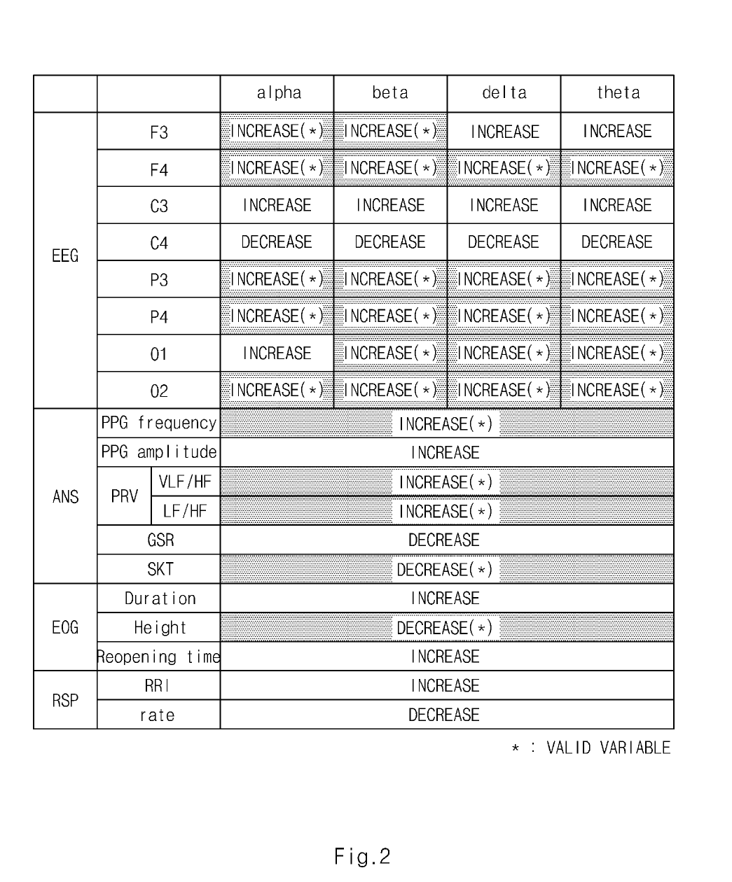

The detailed embodiment of the reference table is described with reference to FIG. 2. The reference table shown in FIG. 2 is a table prepared based on the results drawn from the drowsy driving experiments surveying 31 experimenters.

As the variables applied to the reference table of FIG. 2, there are EEG, PPG, PRV, GSR, SKT, EOG, and RSP. At this time, the EEG measures a state change of alpha, beta, delta, and theta of F3, F4, P3, P4, O1, and O2. The alpha applies a range from 8 to 12.9 Hz, the beta from 13 to 29.9 Hz, the delta from 1 to 3.9 Hz, the theta from 4 to 7.9 Hz, and the PPGs applies a frequency and amplitude. In addition, the PRV applies very low frequency (VLF)/high frequency (HF) and low frequency (LF)/high frequency (HF). In this case, the VLF ranges from 0.0033 to 0.04 Hz, the HF from 0.15 to 0.4 Hz, and the LF from 0.04 to 0.08 Hz.

The GSR and the SKT apply a mean value and the EOG applies a duration, a height, and a reopening time. The RSP applies R wave to R wave interval (RRI) and a rate.

The reference table sets the physiological signal value measured in the awake state as a reference value in the early stage of the experiment of the each experimenter. Thereafter, the reference table is represented as an increase or a decrease according to the degree of the physiological signal value is changed while the user changes to the drowsy state.

In particular, in an exemplary embodiment of the present disclosure, the variables that change in the awake state and drowsy state of the experimenters exceeding standard values, for example, F3 (alpha, beta), F4 (alpha, beta, delta, theta), P3 (alpha, beta, delta, theta), P4 (alpha, beta, delta, theta), O1 (beta, delta, theta), O2 (alpha, beta, delta, theta), PPG frequency, PRV (VLF/HF, LF/HF), SKT, and EOG (height) is defined as valid variables (indicated as ‘*’ in FIG. 2).

The mode determiner 150 sets the operation mode of the apparatus for determining the drowsy state. Here, the operation mode of the apparatus for determining the drowsy state includes a first mode operating only by pre-defined valid variables among whole variables applied to the algorithm for determining the drowsy state and a second mode operating by the whole variables.

The mode determiner 150 may set the operation mode according to an initial setting value and according to a request of a manager or the user.

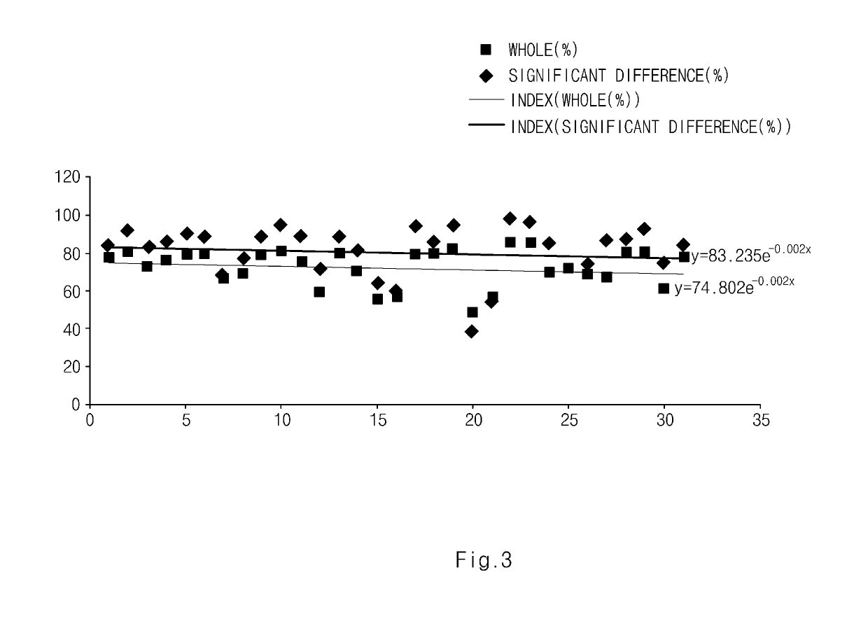

When the operation mode is set by the mode determiner 150, the threshold determiner 160 sets the threshold based on the data in the reference table for the application variables which are applied to the set operation mode. In this case, when the operation mode is set to the first mode by the mode determiner 150, the threshold determiner 160 sets the threshold based on the data in the reference table for the valid variables. Meanwhile, when the operation mode is set to the second mode by the mode determiner 150, the threshold determiner 160 sets the threshold based on the data in the reference table for the whole variables. The exemplary embodiment for the operation setting the threshold will be described in detail with reference to FIG. 3.

When the threshold for the set operation mode is set, the physiological index calculator 170 calculates a current physiological index of the user by analyzing the physiological information of the user acquired in real time by the information acquiring unit 140. In this case, the physiological index calculating unit 170 calculates the physiological index for each of the application variables applied to the operation mode set from the physiological information of the user.

As an example, in a case in which the operation mode is set to the first mode, the physiological index calculator 170 calculates time the physiological index in real for each of the F3 (alpha, beta), F4 (alpha, beta, delta, theta), P3 (alpha, beta, delta, theta), P4 (alpha, beta, delta, theta), O1 (beta, delta, theta), O2 (alpha, beta, delta, theta), PPG frequency, PRV (VLF/HF, LF/HF), SKT, and EOG (height) which are set valid variables.

The state determiner 180 compares the measured value of each variable calculated by the physiological index calculator 170 with a reference value for the corresponding application variable to generate the user table based on a change in the state. Here, the reference value, which is a standard value for the awake state of the user, may be set as a mean value of the value measured for a certain time from the time point of starting to measure the physiological signal of the user. For example, the state determiner 180 determines that the user is in the awake state during 3 minutes from the time point of starting to measure the physiological signal, thereby setting the reference value based on the data for the initial 3 minutes. Meanwhile, the reference value also may be set based on the data measured in the awake state among the data of the reference table.

The state determiner 180 calls the reference table stored in the DB 200 to compare with the user table and determines the drowsy state of the user according to the results of the comparison.

Since the reference data is generated based on the state change value while changing into the drowsy state on the basis of the awake state, the state determiner 180 determines that the user is in the “drowsy” state in the case in which the data of the user table coincides with the data of the reference table. Meanwhile, the state determiner 180 determines that the user is in the “awake” state in the case in which the data of the user table does not coincide with the data of the reference table.

The results of determining the drowsy state of the state determiner 180 may be output through the output unit 130, and an alarm, a warning message, or the like 110 may be output under the control of the controller in the case in which the user is in the drowsy state.

FIG. 3 is a diagram for describing an operation of setting threshold of the each mode based on the reference data. Here, ▪ symbol indicates data of the each experimenter for the whole application variables, and ♦ symbol indicates data of the each experimenter for the valid variable defined through FIG. 2. When the algorithm for determining the drowsy state of the user is operated, the threshold for each mode is set based on the data of each experimenter.

As shown in FIG. 3, — line indicates the threshold at the time of operations in the second mode and — line indicates the threshold at the time of operations in the first mode. In other words, the threshold is calculated based on the data of the each experimenter for the whole application variables at the time of operations in the second mode. The threshold in the second mode calculated from the graph of FIG. 3 is 74.802e-0.002x. Meanwhile, when the apparatus for determining the drowsy state operates in the first mode, only the predefined valid variable becomes the application variable, such that the threshold is calculated based on the data of the each experimenter for the valid variable. The threshold in the first mode calculated from the graph of FIG. 3 becomes 83.235e-0.002x.

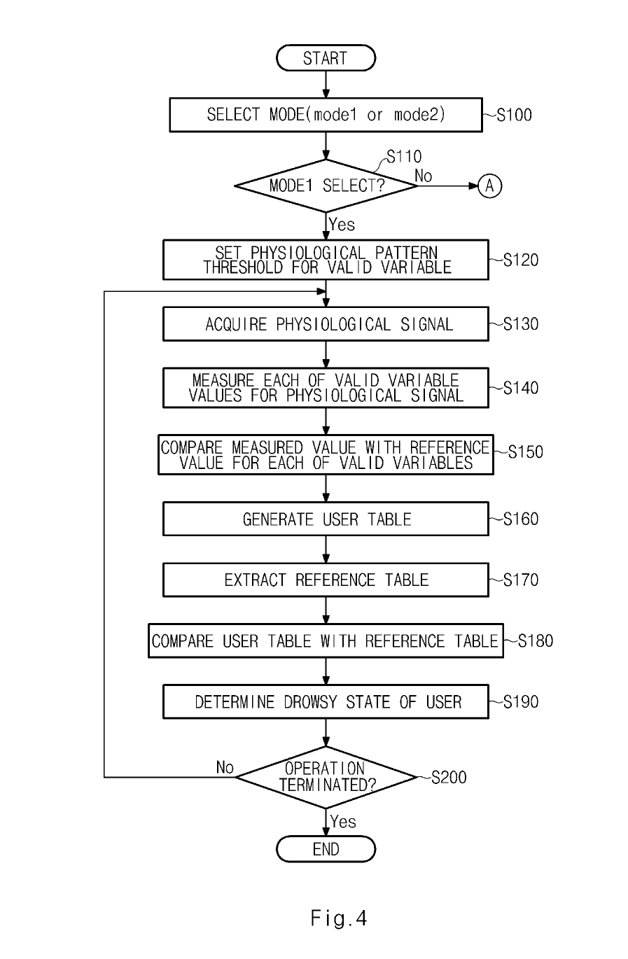

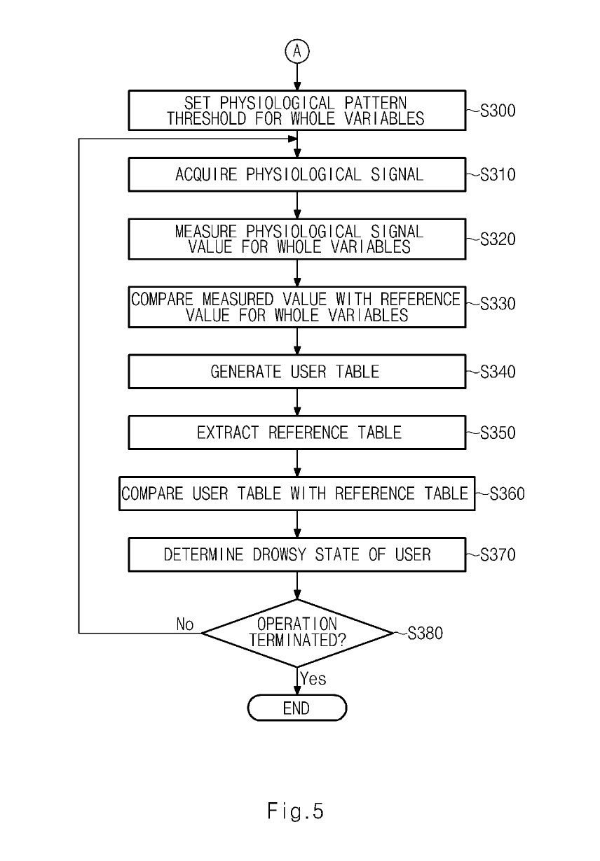

A sequence of the operation of the algorithm for determining the drowsy state in the apparatus for determining the drowsy state according to the exemplary embodiment of the present disclosure is shown in FIG. 4.

Referring to FIG. 4, the apparatus for determining the drowsy state according to the exemplary embodiment of the present disclosure selects an operation mode at the time of execution of the algorithm for determining the drowsy state of the user ridden in the vehicle (S100). In this case, in the operating mode, ‘mode1’ operating only for the predefined valid variables and ‘mode2’ operating for the whole variables may be selected. The operation mode may be added according to the types of embodiment. If the operation mode is selected as ‘mode1’ at step S100 (S110), the apparatus for determining the drowsy state proceeds to step S120. If the operation mode is selected as ‘mode2’, the apparatus for determining the drowsy state enters into (A) and proceeds to step S300.

In other words, when the operation mode is selected as ‘mode1’, the apparatus for determining the drowsy state sets a physiological pattern threshold based on the data for the valid variables in the reference table (S120) and measures each of valid variable values for the physiological signal that is acquired from the user (S130 and S140).

The apparatus for determining the drowsy state compares the value measured for each of the valid variables at step S140 with the reference value set based on the data measured in the awake state of the user (S150) and generates the user table for the user (S160).

Thereafter, the apparatus for determining the drowsy state extracts the reference table from the DB (S170) to compare with the user table generated at step S160 (S180) and determines the drowsy state of the user according to whether the reference table coincides with the user table (S190). Even if the data in the reference table does not accurately coincide with the data in the user table in consideration of an error of measurement, the apparatus for determining the drowsy state may determine that the data in the reference table coincide with each other in the case in which a difference between those data is included in a certain error range.

Steps S130 to S190 are repeatedly executed for the physiological signal of the user measured in real time until the termination of the operation of the algorithm for determining the drowsy state. When an operation termination command is input (S200), the operation terminates.

Meanwhile, when the operation mode is selected as ‘mode2’, the apparatus for determining the drowsy state sets the physiological pattern threshold based on the data for the whole variables of the reference table (S300) and measures the physiological signal value for the whole variable with respect to the physiological signal that is acquired from the user (S310 and S320).

Here, the apparatus for determining the drowsy state compares the value measured for each of the variables at step S320 with the reference value set based on the data measured in the awake state of the user (S330) and generates the user table for the user (S340).

Then, the apparatus for determining the drowsy state extracts the reference table from the DB (S350) to compare with the user table generated at step S340 (S360) and determines the drowsy state of the user according to whether or not the reference table coincides with the user table (S370). Even if the data in the reference table does not accurately coincide with the data in the user table in consideration of an error of measurement, the apparatus for determining the drowsy state may determine that the data in the reference table coincide with each other in the case in which a difference between those data is included in a certain error range.

Steps S310 to S370 are repeatedly executed for the physiological signal of the user measured in real time until the termination of the operation of the algorithm for determining the drowsy state. When the operation termination command is input (S380), the operation terminates.

As set forth above, the exemplary embodiment of the present disclosure can determine the drowsy state of the user by comparing the reference table prepared based on a plurality of users with the user table prepared according to the change in the physiological state of the user.

In addition, the present disclosure comprehensively considers the state change for whole variable or valid variables that may be used for determining the drowsy state of the user, thereby making it possible to precisely determine the drowsy state of the user.

Although exemplary embodiments of the present disclosure have been described in detail hereinabove, it should be clearly understood that many variations and modifications of the basic inventive concepts herein taught which may appear to those skilled in the present art will still fall within the spirit and scope of the present disclosure, as defined in the appended claims.

What is claimed is:

1. An apparatus for determining a drowsy state, the apparatus comprising:
an information acquirer acquiring physiological information of a user;
a mode determiner setting an operation mode among modes categorized according to a variable applied to determine the drowsy state of the user;
a threshold determiner setting a threshold for an application variable of the set mode based on data in a reference table stored in database (DB);
a physiological index calculator calculating a measurement value for each of the application variables based on state information of the user acquired in real time from the information acquirer; and
a state determiner determining the drowsy state of the user by comparing a user table generated from the measurement value calculated for each of the application variables with the reference table.

2. The apparatus of claim 1, wherein the application variable includes at least one among electroencephalogram (EEG), photoplethysmograph (PPG), photoplethysmograph rate variability (PRV), galvanic skin response (GSR), skin temperature (SKT), electrooculography (EOG), and respiration (RSP).

3. The apparatus of claim 1, wherein the reference table is generated according to a change in a physiological signal value measured in the drowsy state based on a physiological signal value measured in a state of awake from a plurality of users with respect to variables applied to determine the drowsy state of the user.

4. The apparatus of claim 1, wherein the user table is generated from results obtained by comparing the measurement value calculated for each of the application variables with a reference value for each of the application variables.

5. The apparatus of claim 4, wherein the reference value is a mean value of the value measured for a given time from a time point of starting to measure a physiological signal of the user.

6. The apparatus of claim 4, wherein the reference value is set based on the data measured in an awake state among the data in the reference table.

7. The apparatus of claim 1, wherein the mode determiner sets any one of a first mode operating only by presetting some valid variables among the application variables and a second mode operating for the whole application variables as the operation mode.

8. The apparatus of claim 7, wherein the valid variable is a variable that has a change of a physiological signal value exceeding a standard value when the physiological signal value is measured in the drowsy state based on the physiological signal value measured in an awake state among the application variables.

9. The apparatus of claim 1, wherein the information acquirer acquires together with action information of the user.

10. A method for determining a drowsy state, the method comprising:
acquiring physiological information of a user;
setting an operation mode among modes categorized according to a variable applied to determine the drowsy state of the user;
setting a threshold for an application variable of the set mode based on data in a reference table stored in database (DB);
calculating a measurement value for each of the application variables based on physiological information acquired in real time from the user;
generating a user table by comparing the measurement value calculated for each of the application variables with a reference value for each of the application variables; and
comparing the reference table called from the DB with the user table and determining the drowsy state of the user according to the comparison result.

11. The method of claim 10, before the acquiring of the physiological information of the user, further comprising:
generating the reference table according to a change of a physiological signal value measured in the drowsy state based on a physiological signal value measured in an awake state from a plurality of users with respect to variables applied to determine the drowsy state of the user.
